(19) United States
(12) Patent Application Publication
(10) Pub. No.: US 2026/0034087 A1
(43) Pub. Date: Feb. 5, 2026

(54) COMPOSITION COMPRISING A TLR4/MD2 PATHWAY INHIBITOR AND AN EGFR INHIBITOR AND ITS USE IN CANCER TREATMENT

(71) Applicant: Hong Kong Baptist University, Hong Kong (CN)

(72) Inventors: Zhiling Yu, Hong Kong (CN); Xiuqiong Fu, Hong Kong (CN); Jingxuan Bai, Hong Kong (CN); Xiaoqi Wang, Hong Kong (CN)

(21) Appl. No.: 19/289,312

(22) Filed: Aug. 4, 2025

Related U.S. Application Data
(60) Provisional application No. 63/679,205, filed on Aug. 5, 2024.

(51) Int. Cl. A61K 31/216; A61K 31/12; A61K 31/45; A61K 31/506; A61K 31/517; A61K 31/7016; A61K 31/7105; A61P 35/00; C07K 16/28

(52) U.S. Cl. CPC A61K 31/216; A61K 31/12; A61K 31/45; A61K 31/506; A61K 31/517; A61K 31/7016; A61K 31/7105; A61P 35/00; C07K 16/2896

(57) ABSTRACT

Disclosed are a composition comprising a TLR4/MD2 pathway inhibitor and an EGFR inhibitor, a pharmaceutical composition comprising the composition, and the use of the composition/pharmaceutical composition in cancer treatment. The TLR4/MD2 pathway inhibitor and the EGFR inhibitor can synergistically kill a variety of cancers, including cutaneous melanomas, and have a significant inhibitory effect on the growth of cancer cells without affecting bodyweight of mice. Moreover, compared with separate administration of the individual components, the composition according to the present disclosure can delay the occurrence of drug resistance in tumor-bearing mice and significantly inhibit the growth of Gefitinib-resistant cancer cells (e.g., non-small cell lung cancer tumors). Therefore, the composition according to the present disclosure can be used for preparing safe and effective anti-tumor medicament.

### CROSS REFERENCE TO RELATED APPLICATIONS

The present application claims priority to the U.S. Provisional Patent Application No. 63/679,205 filed on Aug. 5, 2024, the content of which is incorporated herein by reference in its entirety.

### TECHNICAL FIELD

The present disclosure belongs to the field of biomedicines, and in particular relates to cancer treatment. More particularly, the present disclosure relates to a composition for cancer treatment, which comprises a Toll-like receptor 4 (TLR4)/Myeloid Differentiation Protein 2 (MD2) signal pathway inhibitor and an Epidermal Growth Factor Receptor (EGFR) inhibitor.

### BACKGROUND

Epidermal Growth Factor (EGF) and its receptor EGFR are overexpressed in a variety of human cancers and are related to tumor growth and metastasis. EGFR inhibitors, including Gefitinib, can effectively block EGFR signal transduction, and have been widely used in cancer treatment for almost twenty years, especially in the treatment of kinase-activated EGFR mutated cancers. However, their long-term efficacy is poor as patients may easily develop drug resistance to them, and their efficacy on wild-type EGFR tumors is not ideal. In recent years, studies have been actively made to enhance the efficacy of EGFR inhibitors and delay or overcome the drug resistance thereof. Although EGFR inhibitors and Tyrosine Kinase Inhibitors (TKIs) have been used in clinical combination to treat cancers, but patients developed resistance to this combination therapy shortly afterwards. Combining EGFR inhibitors with radiotherapy, cytotoxic chemotherapy, and other therapies is also suboptimal (e.g., drawbacks such as poor efficacy or increased adverse effects). Breaking through the limitations in the efficacy of EGFR inhibitors remains a significant challenge.

As a transmembrane protein, TLR4 is an important member of the Toll-like receptor family, and is mainly expressed on the surfaces of immune cells and tumor cells. MD2 is a co-receptor of TLR4. The TLR4/MD2 signaling pathway can be activated by ligands such as bacterial lipopolysaccharides (LPS), thereby mediating classical inflammatory responses. Overactivation of the TLR4/MD2 pathway is closely related to a variety of diseases (e.g., septicemias, autoimmune diseases and neuroinflammations). Therefore, the TLR4/MD2 pathway has become an important target for the treatment of inflammation-related diseases. Although some studies have demonstrated that TLR4/MD2 may be involved in cancers, there are few research on the combined application of TLR4/MD2 inhibitors with other therapies, especially EGFR inhibitors.

Therefore, there is still a need to develop a safe and effective compositions for cancer treatment.

### SUMMARY

In a first aspect, the present disclosure provides a composition comprising a TLR4/MD2 pathway inhibitor and an EGFR inhibitor.

In some embodiments, the TLR4/MD2 pathway inhibitor is selected from chemical inhibitors, nucleic acid inhibitors and antibody inhibitors that inhibit the activation of the TLR4/MD2 pathway. In some embodiments, the chemical inhibitors include small molecule inhibitors, such as TAK-242, LPS-RS, E5564, L48H37, MD2-IN-1, and L6H21. In some embodiments, the nucleic acid inhibitors include nucleic acid molecules, such as siRNAs, miRNAs or antisense RNAs. In some embodiments, the antibody inhibitors include monoclonal antibodies, such as anti-TLR4 monoclonal antibodies and anti-MD2 monoclonal antibodies.

In some embodiments, the EGFR inhibitor is selected from chemical inhibitors, nucleic acid inhibitors and antibody inhibitors. In some embodiments, the chemical inhibitors include small molecule inhibitors, such as Gefitinib, Erlotinib, Afatinib, Osimertinib, Dacomitinib, Almonertinib, and Icotinib hydrochloride. In some embodiments, the nucleic acid inhibitors include nucleic acid molecules, such as siRNAs, miRNAs or antisense RNAs. In some embodiments, the antibody inhibitors include monoclonal antibodies, such as Cetuximab, Panitumumab or Necitumumab.

In some embodiments, the EGFR inhibitor comprises Gefitinib, and the TLR4/MD2 pathway inhibitor comprises TAK-242 and/or L48H37. In some embodiments, a mass ratio of the EGFR inhibitor to the TLR4/MD2 pathway inhibitor is about 1:(0.5-3).

In some embodiments, the composition comprises a first composition and a second composition, wherein the first composition comprises a TLR4/MD2 pathway inhibitor, and the second composition comprises an EGFR inhibitor, wherein the first composition and/or the second composition comprise(s) pharmaceutically acceptable excipients. In some embodiments, the first composition comprises a small molecule TLR4/MD2 pathway inhibitor. In some embodiments, the second composition comprises a small molecule EGFR inhibitor.

In some embodiments, the TLR4/MD2 pathway inhibitor is TAK-242 or L48H37, and the EGFR inhibitor is Gefitinib. In some embodiments, a mass ratio of the Gefitinib to the TAK-242 is about 1:1-1:1.5. In some embodiments, a mass ratio of the Gefitinib to the L48H37 is about 1:1-1:1.5.

In a second aspect, the present disclosure provides a pharmaceutical composition comprising the composition described in the first aspect and optionally pharmaceutically acceptable excipients.

In some embodiments, the pharmaceutical composition further comprises an additional anticancer therapeutic agent, which is selected from immunotherapeutic agents, chemotherapeutic agents, antiangiogenic agents, multidrug resistance-related protein inhibitors, radiotherapeutic agents, and any combination thereof.

In a third aspect, the present disclosure provides a method for cancer treatment, comprising administering a therapeutically effective amount of the composition described in the first aspect to a subject in need thereof.

In some embodiments, the cancer is selected from melanomas, eye cancers, oral cancers, lung cancers, liver cancers, bone cancers, brain cancers, gastrointestinal cancers, pancreatic cancers, neurological cancers, urogenital cancers, gynecological cancers, thyroid cancers, adrenal cancers, leukemias, lymphomas, cancers of types resistant to EGFR inhibitors among the above-mentioned cancers, and breast cancers.

In some embodiments, the melanomas are selected from cutaneous melanomas and non-cutaneous melanomas. In some embodiments, the lung cancers are selected from non-small cell lung cancers and small cell lung cancers. In some embodiments, the gastrointestinal cancers are selected from esophageal cancer, gastric cancer, duodenal cancer, small intestine cancer, colon cancer, rectal cancer, colorectal cancer, anal cancer, gallbladder cancer, and cholangiocarcinoma. In some embodiments, the neurological cancers are selected from glioma, meningioma and neurilemmoma. In some embodiments, the urogenital cancers are selected from renal cancer, bladder cancer, urethral cancer, and prostatic cancer. In some embodiments, the gynecological cancers are selected from cervical cancer, endometrial cancer, ovarian cancer, fallopian tube cancer and vaginal cancer.

In some embodiments, the composition is administered by one or more of oral administration, percutaneous administration, intramuscular administration, subcutaneous administration, intraperitoneal administration, and intravenous administration.

In some embodiments, the composition is in a dosage form of tablet, capsule, powder, injection, solution, suspension, emulsion, or any combination thereof.

In some embodiments, the subject is a human or a non-human mammal, including primate such as monkeys, chimpanzees or gorillas; rodent such as mice or rats; bovid; equid; caprid; or canid.

### BRIEF DESCRIPTION OF THE DRAWINGS

It should be understood that the drawings described herein are only for an illustrative purpose. The drawings are not intended to limit the scope of the present disclosure in any way.

FIG. 1 is a photograph of cell clone formation results, showing the effects of inhibiting the TLR4/MD2 or EGFR pathway alone and inhibiting both pathways simultaneously on the proliferation of melanoma cells. (A) Effects of inhibiting the TLR4 or EGFR pathway alone and inhibiting both pathways on the proliferation of A2058 cells. EGFR^{-KO} represents A2058 human melanoma cells with EGFR knockout; TLR4^{-KO} represents A2058 cells with TLR4 knockout; and EGFR^{-KO} TLR4^{-KO} represents A2058 cells with EGFR-TLR4 double genes knockout; EGFRi (EGFR inhibitor) represents Gefitinib at 2.5 μM; TLR4i (TLR4 inhibitor) represents TAK-242 at 5 μM. (B) Effects of inhibiting the MD2 or EGFR pathway alone and inhibiting both pathways on the proliferation of A375 cells. MD2i (MD2 inhibitor) represents L48H37 at 0.1 μM.

FIG. 2 shows the effects of using the EGFR inhibitor and the TLR4/MD2 pathway inhibitor separately and in combination on the survival rate of the tumor cells. * indicates that a coefficient of drug interaction (CDI) of the two drugs is smaller than 1, suggesting a synergistic effect of the drug combination; and ** indicates that the CDI is smaller than 0.7, suggesting a significant synergistic effect.

FIG. 3 shows the effects of using the EGFR inhibitor and the TLR4/MD2 pathway inhibitor separately and in combination on the growth of subcutaneous melanomas in mice. (A) images of tumors excised from mice and the weights of the tumors; (B) tumor volumes of the mice; and (C) body weights of the mice; * indicates P<0.05 compared with the control group.

FIG. 4 shows the inhibitory effect of combination of EGFR inhibitor and TLR4/MD2 pathway inhibitors on the growth of a Gefitinib-resistant non-small cell lung cancer tumors in a mouse model. (A) images of tumor-bearing mice; (B) quantitative analysis of tumor fluorescence intensity in the mice; * indicates P<0.05 compared with the control group.

FIG. 5 shows the incidence of drug resistance in tumor-bearing mice treated with EGFR inhibitor alone, TLR4/MD2 pathway inhibitor alone, or their combination.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers.

The term “essentially consisting of” and the like permit the existence of components/elements not explicitly enumerated, but exclude components/elements existing in the prior art or components/elements that affect the essence or novelty of the present disclosure.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a composition/pharmaceutical composition described herein, then the subject has been the object of treatment, observation, and/or administration of the composition/pharmaceutical composition described herein.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

The term “pharmaceutically acceptable” means that the components of a pharmaceutical composition are compatible with each other and are not harmful to the administered subject. Similarly, the term “pharmaceutically acceptable excipient” refers to a substance that does not produce unfavorable, allergic or other adverse reactions when administered to humans or other mammals. In some embodiments, excipients include carriers, diluents, excipients and/or adjuvants. For human administration, pharmaceutical compositions or preparations shall conform to the criteria for sterility, general safety and purity as specified by regulatory authorities (e.g., NMPA or FDA).

The terms “application”, “administration” or similar expressions refer to the provision of an active agent or active ingredient (e.g., a TLR4/MD2 pathway inhibitor and/or an EGFR inhibitor) to a subject in need of treatment, either alone or as part of a pharmaceutically acceptable composition.

The term “optional (ly)” herein means that the feature (e.g., component, step, etc.) defined by the term may or may not be present. When the term is used in the claims and the specification, in the case where it indicates the “absence” of a defined feature, it should be interpreted as the claims do not define the feature, rather than that the claims exclude the presence of the feature. For example, when the claims define “optionally, the pharmaceutical composition further comprises an additional anticancer therapeutic agent”, it involves two situations. The first situation indicates that the claims define “pharmaceutical composition further comprises an additional anticancer therapeutic agent”, and the other situation indicates that the claims do not specifically define “pharmaceutical composition further comprises an additional anticancer therapeutic agent”, but do not exclude the existence of this feature, unless otherwise indicated in the context.

As used herein, the term “TLR4/MD2 pathway inhibitor” refers to an inhibitor capable of blocking TLR4/MD2 signaling (e.g., by interfering with the binding of TLR4 to MD2 and/or inhibiting its downstream signaling processes). In certain embodiments, TLR4/MD2 pathway inhibitors include TLR4 inhibitors and MD2 inhibitors, provided they can effectively block this signaling pathway. A “TLR4 inhibitor” refers to a substance that reduces TLR4-mediated inflammatory responses or abnormal activation by directly binding to the TLR4 protein, blocking its binding to ligands (such as LPS), inhibiting TLR4 homodimerization, or interfering with its intracellular signal transduction. An “MD2 inhibitor” refers to a substance that inhibits the formation or activation of the TLR4-MD2 complex by binding to MD2 and blocking its interaction with ligands (such as LPS) or TLR4.

As used herein, the term “EGFR inhibitors” refer to a class of drugs that inhibit the proliferation of tumor cells and induce apoptosis by blocking the activation of EGFRs or the downstream signal transduction.

As used herein, the term “miRNA” refers to a synthetic miRNA, unless otherwise indicated.

As used herein, the terms “chemical preparation,” “nucleic acid preparation,” “antibody preparation,” or similar expressions are intended to refer to preparations where the active ingredient or main active ingredient is a compound (e.g., a small-molecule compound), a nucleic acid molecule, or an antibody. In addition to the active ingredient, such preparations may contain inactive substances that do not affect the activity of the active ingredient and are harmless to the subject, such as pharmaceutically acceptable excipients (e.g., carriers, diluents, excipients, and/or adjuvants, etc.). For example, a “nucleic acid preparation” refers to a preparation containing a nucleic acid molecule as the active ingredient or main active ingredient, which may additionally include pharmaceutically acceptable excipients; and so on. In some embodiments, “preparation” and “composition” may be used interchangeably.

In this specification, when an expression “the pharmaceutical composition comprises the composition described in this disclosure and optionally pharmaceutically acceptable excipients” or a similar expression is mentioned, it should be understood that “optionally” is defined as above, and, in the case that the composition comprised in the pharmaceutical composition (e.g., the composition described in the first aspect of the present disclosure) already contains pharmaceutically acceptable excipients, it is unnecessary for the pharmaceutical composition to contain additional pharmaceutically acceptable excipients, though this situation is not excluded.

Unless otherwise specified, singular forms used herein encompass corresponding plural forms (and vice versa).

As used herein, the term “about”, when used before a quantitative value, also includes the stated quantitative value itself, unless explicitly stated otherwise. As used herein, the term “about” means a variation of +/−10%, +/−7%, +/−5%, +/−3%, +/−1% or +/−0% compared with the recited value or range, unless explicitly stated otherwise.

The present disclosure provides a composition having an anticancer effect, the active ingredient of which comprises or consists of a TLR4/MD2 pathway inhibitor and an EGFR inhibitor. In the composition provided in the present disclosure, the TLR4/MD2 pathway inhibitor and the EGFR inhibitor achieve a significant synergistic anticancer effect, and have an excellent inhibitory effect on wild-type EGFR tumors and kinase-activated EGFR mutated tumors. In addition, the composition in the present disclosure can effectively delay or overcome the drug resistance of tumor-bearing animals to EGFR inhibitors.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

In a first aspect, the present disclosure provides a composition comprising a TLR4/MD2 pathway inhibitor and an EGFR inhibitor.

In some embodiments, the active ingredient of the composition comprises or consists of a TLR4/MD2 pathway inhibitor and an EGFR inhibitor.

In some embodiments, the TLR4/MD2 pathway inhibitor is selected from chemical inhibitors, nucleic acid inhibitors and antibody inhibitors, as long as it can block TLR4/MD2 signal transduction or inhibit the activation of a TLR4/MD2 pathway. In some embodiments, the chemical inhibitors include polypeptides (e.g., TLR4/MD2 binding peptides) and small molecule inhibitors, such as TAK-242 (Resatorvid), LPS-RS, E5564, L48H37, MD2-IN-1, or L6H21. In some embodiments, the nucleic acid inhibitors include nucleic acid molecules, such as siRNAs (e.g., siRNAs targeting the TLR4 gene and siRNAs targeting the MD2 gene), miRNAs (e.g., miRNA-146a) or antisense RNAs (e.g., antisense RNAs targeting TLR4 or MD2). In some embodiments, the antibody inhibitors include monoclonal antibodies, such as anti-TLR4 monoclonal antibodies (e.g., HTA125) and anti-MD2 monoclonal antibodies (e.g., MTS510).

In some embodiments, the TLR4/MD2 pathway inhibitor is selected from a TLR4 inhibitor (TLR4i), a MD2 inhibitor (MD2i), and a combination thereof. In some embodiments, the TLR4 inhibitor is selected from TAK-242, CU-CPT22, Felvizumab, T5342126, FP7, CLI-095, Eritoran, and any combination thereof. In some embodiments, the MD2 inhibitor is selected from FPS-ZM1, SPL7073, L6H21, T6071187, L48H37, and any combination thereof.

In some embodiments, the EGFR inhibitor is selected from chemical inhibitors, nucleic acid inhibitors and antibody inhibitors, as long as it can inhibit the activation of EGFRs or the downstream signal transduction. In some embodiments, the EGFR inhibitor is selected from chemical inhibitors, nucleic acid inhibitors or antibody inhibitors that inhibit EGFRs. In some embodiments, the chemical inhibitors include small molecule inhibitors, such as Gefitinib, Erlotinib, Afatinib, Osimertinib, Dacomitinib, Almonertinib, and Icotinib hydrochloride. In some embodiments, the nucleic acid inhibitors include nucleic acid molecules, such as siRNAs, miRNAs and antisense RNAs. In some embodiments, the antibody inhibitors include monoclonal antibodies, such as Cetuximab, Panitumumab and Necitumumab.

In some embodiments, the EGFR inhibitor is selected from a tyrosine kinase inhibitor (TKI), an anti-EGFR monoclonal antibody, or a combination thereof. In some embodiments, the TKI is selected from Gefitinib, Erlotinib, Osimertinib, or a combination thereof. In some embodiments, the anti-EGFR monoclonal antibody is selected from Cetuximab, Panitumumab, or a combination thereof.

In some embodiments, the composition comprises or consists of Gefitinib and TAK242.

In some embodiments, the composition comprises or consists of Gefitinib and L48H37.

In some embodiments, a mass ratio of the EGFR inhibitor (e.g., Gefitinib) to the TLR4/MD2 pathway inhibitor (e.g., TAK242 or L48H37) is about 1:(0.5-3), such as about 1:(0.75-2), 1:(0.75-1), 1:(1-3), 1:(1-1.5), 1:0.75, 1:1.3, 1:1.5, or any value or range therebetween. In some embodiments, a mass ratio of the EGFR inhibitor Gefitinib to the TLR4 inhibitor TAK242 is about 1:(0.5-3), such as 1:(0.75-2), 1:(0.75-1), 1:1, or any value or range therebetween. In some embodiments, a mass ratio of the EGFR inhibitor Gefitinib to the MD2 inhibitor L48H37 is about 1:(0.5-3), such as 1:(1-1.5), 1:1.3, 1:1.5, or any value or range therebetween.

In some embodiments, the composition comprises a first composition and a second composition, wherein the first composition comprises a TLR4/MD2 pathway inhibitor (e.g., as the sole active ingredient), and the second composition comprises an EGFR inhibitor (e.g., as the sole active ingredient). In some embodiments, the first composition and the second composition comprise pharmaceutically acceptable excipients respectively. In some embodiments, the excipients in the first composition may be the same as or different from the excipients in the second composition. In some embodiments, the first composition is a chemical preparation comprising a TLR4/MD2 pathway chemical inhibitor, a nucleic acid preparation comprising a TLR4/MD2 pathway nucleic acid inhibitor, or an antibody preparation comprising a TLR4/MD2 pathway antibody inhibitor. In some embodiments, the first composition is a chemical preparation comprising a small molecule TLR4/MD2 pathway inhibitor (e.g., TAK-242 and/or L48H37). In some embodiments, the second composition is a chemical preparation containing a chemical EGFR inhibitor, a nucleic acid preparation containing a nucleic acid-based EGFR inhibitor, or an antibody preparation containing an antibody-based EGFR inhibitor. In some embodiments, the second composition is a chemical preparation comprising a small-molecule EGFR inhibitor, such as Gefitinib. In some embodiments, the first composition comprises TAK-242 and/or L48H37 as the sole active ingredient. In some embodiments, the first composition consists of TAK-242 and/or L48H37 and pharmaceutically acceptable excipients. In some embodiments, the second composition comprises Gefitinib as the sole active ingredient. In some embodiments, the second composition consists of Gefitinib and pharmaceutically acceptable excipients. In some embodiments, the composition consists of the first composition and the second composition.

In some embodiments, the first composition and the second composition are packaged separately.

In some embodiments, the first composition comprises about 0.5-10 mg/ml (e.g., about 1-8 mg/ml, 1.2-5 mg/ml, 1.5-3.5 mg/ml, 1.6-3 mg/ml, 1.8-2.5 mg/ml, 2 mg/ml, or any number or range therebetween) TLR4/MD2 pathway inhibitor (e.g., TAK-242 or L48H37).

In some embodiments, the second composition comprises about 0.1-5 mg/ml (e.g., about 0.2-4 mg/ml, 0.3-3 mg/ml, 0.5-2.5 mg/ml, 0.6-2 mg/ml, 0.7-1.5 mg/ml, 0.75 mg/ml, or any number or range therebetween) EGFR inhibitor (e.g., Gefitinib).

In a second aspect, the present disclosure provides a pharmaceutical composition comprising the composition described in the first aspect and optionally pharmaceutically acceptable excipients.

In some embodiments, the pharmaceutical composition further comprises an additional anticancer therapeutic agent, which is selected from immunotherapeutic agents, chemotherapeutic agents, antiangiogenic agents, multidrug resistance-related protein inhibitors, radiotherapeutic agents, and any combination thereof.

In some embodiments, the immunotherapeutic agents are selected from checkpoint inhibitors, checkpoint agonists, IDO inhibitors, PI3K inhibitors, adenosine receptor inhibitors, adenosine-producing enzyme inhibitors, CD40 agonists, IL2 variants, immune cells, therapeutic vaccines, or any combination thereof. For example, the checkpoint inhibitors may be selected from inhibitors for checkpoint proteins of PD-1, PD-L1, CTLA-4 and TIGIT.

In some embodiments, the chemotherapeutic agents are selected from anticancer alkylating agents, anticancer antimetabolites, anticancer antibiotics, anticancer agents of plant origin, anti-hormone-sensitive cancer agents, and any combination thereof. For example, the chemotherapeutic agents may be selected from Adriamycin, Oxaliplatin, Fluorouracil, Oxaliplatin, Paclitaxel, Gemcitabine, Nab-Paclitaxel, and any combination thereof.

In some embodiments, the additional anticancer therapeutic agent is selected from chemotherapeutic drugs (e.g., Gemcitabine, Cisplatin, Paclitaxel, Doxorubicin, 5-Fluorouracil, etc.), targeted therapeutic drugs (VEGF inhibitors, ALK inhibitors, PD-1/PD-L1 monoclonal antibodies, CTLA-4 inhibitors, etc.), endocrine therapeutic drugs (e.g., Tamoxifen, Letrozole, Abiraterone, etc.), immunomodulators (e.g., interferon, interleukin-2, etc.), radiotherapeutic sensitizers or other biological preparations with anti-tumor activity, and any combination thereof.

In some embodiments, the additional anticancer therapeutic agent is included in a third composition. In some embodiments, the third composition further comprises pharmaceutically acceptable excipients.

In some embodiments, the first composition, the second composition, and the third composition are packaged separately.

In a third aspect, the present disclosure provides the use of the composition or pharmaceutical composition according to the present disclosure in the preparation of a medicament for treating diseases of a subject, in particular for treating cancer.

In a fourth aspect, the present disclosure provides a method for treating diseases, e.g., cancer, of a subject, which comprises administering a therapeutically effective amount of the composition or pharmaceutical composition described in the present disclosure to the subject.

In a fifth aspect, the present disclosure provides a kit comprising the composition or pharmaceutical composition described in the present disclosure and an optional instruction for using the composition or pharmaceutical composition.

In some embodiments in various aspects, the disease is selected from cancer, inflammatory diseases, autoimmune diseases, metabolic diseases, neurodegenerative diseases, or any combination thereof.

In some embodiments, the disease is cancer. In some embodiments, the cancer is selected from melanomas (e.g., cutaneous melanomas, non-cutaneous melanomas, uveal tract melanomas), eye cancers, oral cancers, lung cancers (e.g., non-small cell lung cancers), liver cancers, bone cancers, brain cancers, gastrointestinal cancers (e.g., colorectal cancer, gastric cancer), pancreatic cancers, neurological cancers (e.g., glioma, neuroglioma), urogenital cancers, and gynecological cancers (e.g., cervical cancer, endometrial cancer, ovarian cancer), thyroid cancers, adrenal cancers, leukemias, lymphomas and breast cancer. In some embodiments, the cancer is that resistant to EGFR inhibitors among the above-mentioned cancers.

In some embodiments, the melanomas include but are not limited to cutaneous melanomas or non-cutaneous melanomas, such as mucosal melanoma, ocular melanomas and uveal tract melanomas. In some embodiments, the lung cancers include but are not limited to non-small cell lung cancers (e.g., lung adenocarcinoma, squamous cell lung carcinoma, large cell lung cancer) or small cell lung cancers. In some embodiments, the gastrointestinal cancers include but are not limited to esophageal cancer, gastric cancer, duodenal cancer, small intestine cancer, colon cancer, rectal cancer, colorectal cancer, anal cancer, gallbladder cancer, or cholangiocarcinoma. In some embodiments, the neurological cancers include but are not limited to glioma, meningioma and neurilemmoma. In some embodiments, the urogenital cancers include but are not limited to renal cancer, bladder cancer, urethral cancer, and prostatic cancer. In some embodiments, the gynecological cancers include but are not limited to cervical cancer, endometrial cancer, ovarian cancer, fallopian tube cancer and vaginal cancer.

In some embodiments, the inflammatory diseases include but are not limited to sepsis, inflammatory bowel diseases or psoriasis. In some embodiments, the autoimmune diseases include rheumatoid arthritis and systemic lupus erythematosus. In some embodiments, the metabolic diseases include diabetes and atherosclerosis. In some embodiments, the neurodegenerative diseases include but are not limited to Alzheimer's disease.

In some embodiments in various aspects, the first composition, the second composition and/or the third composition are administered separately, simultaneously or sequentially. For example, when two compositions are administered, the first composition and the second composition are administered simultaneously; alternatively, the first composition is administered first, and then the second composition is administered; alternatively, the second composition is administered first, and then the first composition is administered. For example, when three compositions are administered, the third composition may be administered before, after or between the first composition and/or the second composition.

In some embodiments in various aspects, the composition or pharmaceutical composition is administered by one or more of oral administration, percutaneous administration, intramuscular administration, subcutaneous administration, intraperitoneal administration, and intravenous administration. In some embodiments, the first composition, the second composition and/or the third composition are administered individually and independently by oral administration (including intragastric administration), percutaneous administration, intramuscular administration, subcutaneous administration, intraperitoneal administration and intravenous administration. In some embodiments, the first composition is administered by oral administration, intraperitoneal administration or intravenous administration. In some embodiments, the second composition is administered by oral administration (including intragastric administration) or intravenous administration.

In some embodiments in various aspects, the composition or pharmaceutical composition is in a dosage form of tablet, capsule, powder, injection, solution, suspension, emulsion, or any combination thereof. In some embodiments, the dosage forms of the first composition, the second composition and/or the third composition are independently selected from tablet, capsule, powder, injection, solution, suspension, emulsion, or any combination thereof. In some embodiments, the dosage form of the first composition is tablet or injection. In some embodiments, the dosage form of the second composition is tablet, solution or suspension.

In some embodiments in various aspects, the subject is a human or a non-human mammal, including primate (e.g., monkey, chimpanzee or gorilla), rodent (e.g., mouse or rat), bovid, equid, caprid or canid.

In some embodiments in various aspects, the excipients include pharmaceutically acceptable carriers, diluents, vehicles, adjuvants, or any combination thereof. In some embodiments, the excipients include one or more of carriers, diluents, vehicles and adjuvants. In some embodiments, the excipients include any inactive substance, such as solvents, cosolvents, antioxidants, surfactants, stabilizers, emulsifiers, buffers, pH regulators, preservatives, antibacterial and antifungal agents, isotonic agents, granulators or adhesives, lubricants, disintegrants, glidants, diluents or fillers, adsorbents, dispersants, suspension agents, coating agents, bulking agents, release agents, absorption retarders, sweeteners, flavoring agents, etc., or any combination thereof.

Unless otherwise stated or contradicting to the common knowledge in the art, one or more features described in the above embodiments of the present disclosure can be combined with each other in any way.

Compared with the prior art, the composition provided by the present disclosure has at least the following beneficial technical effects:
(1) By combining TLR4/MD2 pathway inhibitor and an EGFR inhibitor, they can achieve a synergistic anti-cancer effect. Their inhibitory effect on tumor growth is significantly better than that of administering either inhibitor alone.
(2) The composition provided by the present disclosure has an excellent inhibitory effect on wild-type EGFR tumors and kinase-activated EGFR mutated tumors, and overcomes the problem of poor efficacy of existing EGFR tyrosine kinase inhibitors in treating EGFR wild-type tumors.
(3) The composition provided by the present disclosure can reverse tumor cell resistance to EGFR inhibitors, delay the occurrence of drug resistance, and prolong the effective duration of the drugs.

### EXAMPLES

The present disclosure will be further described below in connection with specific examples, so that those skilled in the art can fully understand the present disclosure. However, it should be noted that these examples are only for an illustrative purpose, but are not intended to limit the scope of protection of the present disclosure. Any modification, equivalent substitution or variation based on the technical concept of the present disclosure should be deemed as falling in the scope of protection of the present disclosure.

### Example 1. Effect of Simultaneously Inhibiting TLR4/MD2 and EGFR Pathway on the Proliferation of Melanoma Cells

### 1.1 Experimental Materials

(1) TAK-242: purchased from MedChemExpress (MCE), catalog number HY-11109; an appropriate amount of TAK-242 was weighed and dissolved into dimethyl sulfoxide (DMSO) to prepare a 50 mM TAK-242 solution (mother solution), then the solution was sub-packaged, and stored in a refrigerator at −20° C. for later use.
(2) L48H37: purchased from MCE, catalog number HY-126154; an appropriate amount of L48H37 was weighed and dissolved into DMSO to prepare a 40 mM L48H37 solution (mother solution), then the solution was sub-packaged, and stored in a refrigerator at −20° C. for later use.
(3) Gefitinib: purchased from MCE, catalog number HY-50895; an appropriate amount of Gefitinib was weighed and dissolved into DMSO to prepare a 50 mM Gefitinib solution (mother solution), then the solution was sub-packaged, and stored in a refrigerator at −20° C. for later use.
(4) EGFR CRISPR double nickase plasmid: purchased from Santa Cruz, catalog number sc-400015-NIC.
(5) TLR4 CRISPR double nickase plasmid: purchased from Santa Cruz, catalog number sc-400068-NIC.
(6) Control double nickase plasmid: purchased from Santa Cruz, catalog number sc-437281.
(7) A375 cell strain (human melanoma cells carrying wild-type EGFRs): purchased from American Type Culture Collection (ATCC), stored in liquid nitrogen.
(8) A2058 cell strain (human melanoma cell with low expression of wild-type EGFRs): purchased from American Type Culture Collection (ATCC), stored in liquid nitrogen.
(9) A2058 cells with EGFR (EGFR^{-KO}) or TLR4 (TLR4^{-KO}) knockout, A2058 cells with EGFR and TLR4 (EGFR^{-KO} TLR4^{-KO}) knockout, and control A2058 cells: The EGFR CRISPR double nickase plasmid or/and the TLR4 CRISPR double nickase plasmid, or the control double nickase plasmid was transfected into A2058 cells. After 48 hours, the cells are screened with 25 μg/mL puromycin. Monoclonal cells were expanded and identified. Then, A2058 cells confirmed to have single knockout of EGFR or TLR4 and those with double knockout of EGFR and TLR4 were selected for subsequent experiments.
(10) DMEM medium powder: from Gibco (USA).
(11) 0.25% trypsin-EDTA (Trypsin): 500 mL/bottle, from Gibco (USA).
(12) Fetal bovine serum (FBS): 500 mL/bottle, from Gibco (USA).
(13) Six-well flat-bottom culture plate and 60 mm petri dish: from SPL (South Korea).
(14) Crystal violet powder: purchased from MERCK, catalogue number Sigma Aldrich C0775.

### 1.2 Experimental Instruments

(1) Carbon dioxide incubator: Model nu-4750e, from Nuaire.
(2) Centrifuge: Model 5702, from Eppendorf.
(3) Biosafety cabinet: Model nu-425-400e, from Nuaire.
(4) Electronic scale: from Mettler Toledo.
(5) Inverted microscope: from Leica.
(6) Water bath: Model WNB 14, from Memmert GmbH+Co.

### 1.3 Experimental Method and Result

Preparation of culture medium: DMEM medium powder was weighed, dissolved in ultra-pure water, filtered and sterilized; then 10% FBS was added, and the mixture was mixed thoroughly to prepare a DMEM culture medium.

Cell passage: A375, EGFR^{-KO} A2058, TLR4^{-KO} A2058, EGFR^{-KO} TLR4^{-KO} A2058 and control A2058 were treated and then inoculated into the DMEM culture medium respectively. Incubate the cells in a carbon dioxide incubator at 37° C. with 5% CO2 and saturated humidity. For cell passage, use 0.25% trypsin-EDTA digestion solution and passage the cells every 2-3 days.

Cell pretreatment: The cultured A375, EGFR^{-KO} A2058, TLR4^{-KO} A2058, EGFR^{-KO} TLR4-KO A2058 and control A2058 cells were collected. The concentrations of the cell suspensions were adjusted to 500 cells/mL. The cell suspensions were added into a six-well culture plate (2 mL/well) or a 60 mm petri dish (3 mL/dish), and incubated in a carbon dioxide incubator at 37° C. with 5% CO2 and saturated humidity for 24 hours.

Drug treatment: For A2058 cells (including EGFR^{-KO} A2058, TLR4^{-KO} A2058, EGFR^{-KO} TLR4^{-KO} A2058, and control A2058 cells): Aspirate the old culture medium from the culture plate/dish. DMEM culture mediums containing 0.1% DMSO (vehicle control), 2.5 μM EGFR inhibitor (EGFRi, i.e., Gefitinib) and/or 5 μM TLR4 inhibitor (TLR4i, i.e., TAK242) were added to the culture plate/dish respectively. Continue culture for 14 days.

For A375 cells: Aspirate the old medium. DMEM culture mediums containing 0.1% DMSO (vehicle control), 10 UM EGFR inhibitor (Gefitinib) or 0.1 μM MD2 inhibitor (L48H37) were added respectively. Continue culture for 28 days.

During the drug treatment, the cell culture mediums were replaced once every three days.

Crystal violet staining: After the experiment, the culture mediums were discarded, and the cells were washed with PBS, and then an appropriate amount of crystal violet dye solution was added for staining. Excessive dye was removed by washing. Cell morphology and clone formation were observed and recorded with an inverted microscope.

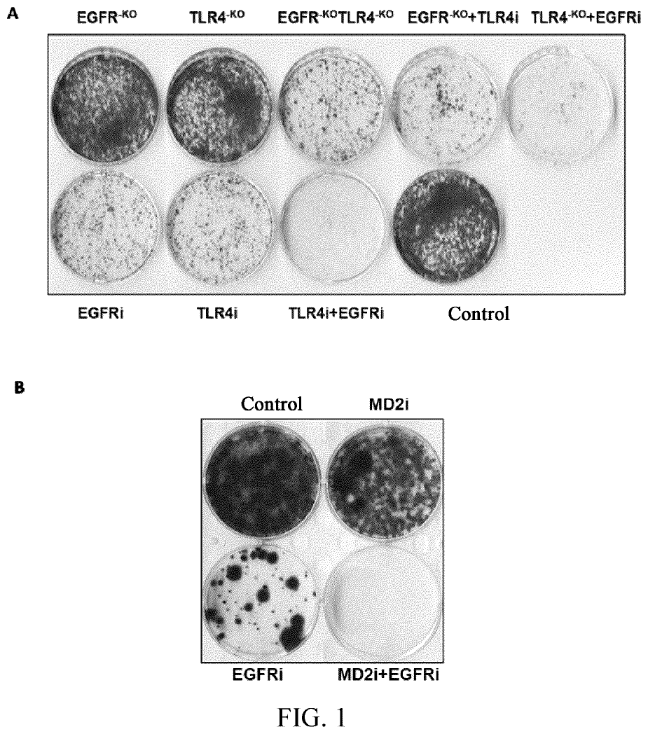

Experimental result: As shown in FIG. 1, the proliferation of A2058 melanoma cells with both TLR4 and EGFR knockout slowed down obviously compared with cells with TLR4 or EGFR knockout. When an TLR4 inhibitor was added to the cells with EGFR knockout or an EGFR inhibitor was added to the cells with TLR4 knockout, it was found that the proliferation capacity of the cells was weaker than that of the control cells that was treated with the TLR4 inhibitor or the EGFR inhibitor alone without gene knockout. Moreover, when a TLR4 inhibitor (TAK242) or MD2 inhibitor (L48H37) was used in combination with an EGFR inhibitor (Gefitinib), it was found that the inhibitory effect on cell proliferation was much stronger than either TLR4/MD2 inhibitor or the EGFR inhibitor alone.

### Example 2: Effect of EGFR Inhibitor+TLR4/MD2 Pathway Inhibitor on the Survival Rate of Tumor Cells

### 2.1 Experimental Materials

(1) TAK-242: purchased from MCE, catalogue number HY-11109; an appropriate amount of TAK-242 was weighed and dissolved into DMSO to prepare 200 mM TAK-242 solution (mother solution), then the solution was sub-packaged, and stored in a refrigerator at −20° C. for later use.
(2) L48H37: purchased from MCE, catalogue number HY-126154; an appropriate amount of L48H37 was weighed and dissolved into DMSO to prepare 40 mM L48H37 solution (mother solution), then the solution was sub-packaged, and stored in a refrigerator at −20° C. for later use.
(3) Gefitinib: purchased from MCE, catalogue number HY-50895; an appropriate amount of Gefitinib was weighed and dissolved into DMSO to prepare 100 mM Gefitinib solution (mother solution), then the solution was sub-packaged, and stored in a refrigerator at −20° C. for later use.
(4) Tumor cell strains carrying wild-type EGFRs A375, Hs294T, A2058, SH-4 cutaneous melanoma cells, mel-202 uveal tract melanoma cells, MIA-PaCa-2 pancreatic cancer cells, T98G, U251 glioma cells, HepG2 and Huh 7 liver cancer cells were purchased from ATCC and stored in liquid nitrogen. An A549 cell strain carrying wild-type EGFRs and a PC9 non-small cell lung cancer cell strain carrying kinase-activated EGFR mutations were purchased from the Cell Bank of China Academy of Sciences and stored in liquid nitrogen.
(6) 0.25% trypsin-EDTA (Trypsin): 500 mL/bottle, from Gibco (USA).
(7) Fetal bovine serum (FBS): 500 mL/bottle, from Gibco (USA).
(8) 96-well flat-bottom culture plate: from SPL (South Korea).
(9) CCK8 kit: from MCE, in trade name of HY-K0301.

### 2.2 Experimental Instruments

The instruments were the same as those described in “1.2 Experimental instruments” in Example 1, except that a microplate reader (manufactured by Thermo Fisher) was used.

### 2.3 Experimental Method and Results

Preparation of culture medium: DMEM medium powder was weighed, dissolved in ultra-pure water, filtered and sterilized; then 10% FBS was added, and the mixture was mixed thoroughly to prepare a DMEM culture medium.

Cell passage: The tumor cell strains (including A375, Hs294T, A2058, SH-4, mel-202, MIA-PaCa-2, T98G, U251, HepG2, Huh7, A549 and PC9) were inoculated into the prepared DMEM medium, and incubated in a carbon dioxide incubator at 37° C. with 5% CO2 and saturated humidity. The cells were passaged by treating with 0.25% trypsin-EDTA digestive solution once every two to three days.

Cell pretreatment: the cultured tumor cells were collected, and the concentration of the cell suspension was adjusted to 3×10⁴ cells/mL. The cells were inoculated into a 96-well flat-bottom culture plate, with 100 μL cell suspension added to each well. Then the culture plate was incubated in a cell incubator at 37° C. with 5% CO2 and saturated humidity for 24 hours.

Drug treatment: Aspirate the old culture medium, and add DMEM culture media separately comprising the following drugs at different concentrations respectively: TAK-242 (TLR4 inhibitor); L48H37 (MD2 inhibitor); Gefitinib (EGFR inhibitor); TAK-242+Gefitinib; or L48H37+Gefitinib. Include a control group (0.1% DMSO) and blank wells (no cell). Set up triplicate wells for reach group and continue culturing for 48 hours.

Cell viability test: after drug treatment, 10 μL CCK8 reagent was added to each well.

Incubation for 1-3 hours, the optical density (OD) in each well was measured with a microplate reader at a wavelength of 450 nm.

Cell survival rate and drug combination effects were calculated using the following formulas:

Cell Survival Rate (%)=(OD value of the drug treatment group−OD value of the blank well)/(OD value of the control group−OD value of the blank well)*100;

Coefficient of Drug Interaction (CDI)=(survival rate of drug combination group/survival rate of the control group)/([(survival rate of the TAK-242 or L48H37 group/survival rate of control group)*(survival rate of the Gefitinib group/survival rate of control group)], where CDI>1 indicates that the two drugs have an antagonistic effect; CDI=1 indicates that the two drugs have an additive effect; CDI<1 indicates that the two drugs have a synergistic effect. For example, CDI<0.7 indicates that the two drugs have a significant synergistic effect.

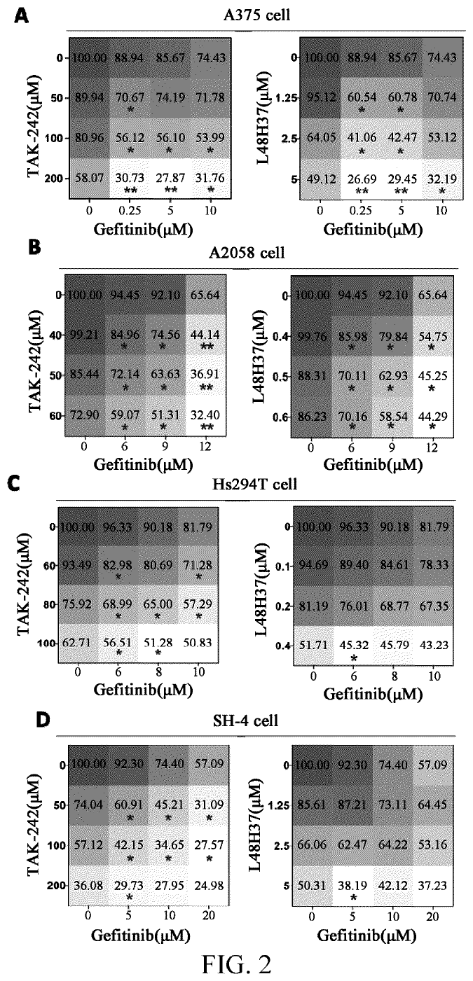

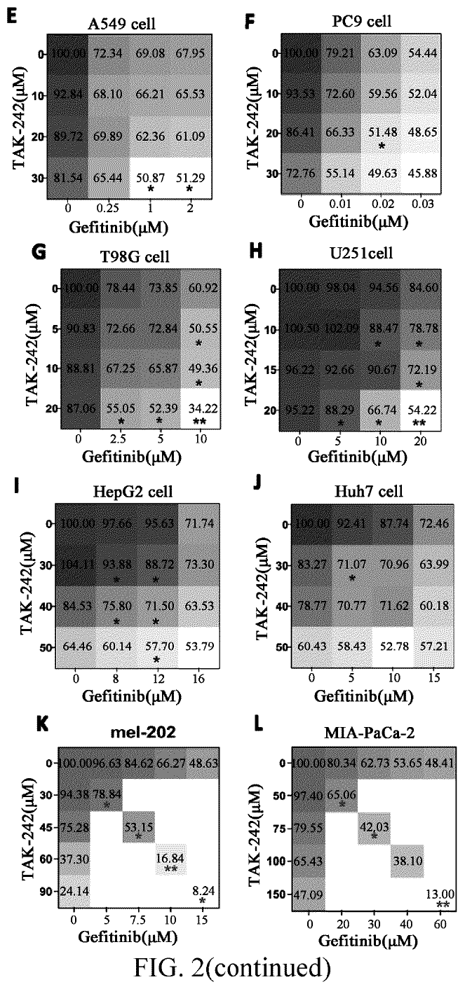

Experimental result: As shown in FIG. 2, combination use of EGFR inhibitor and the TLR4/MD2 pathway inhibitor killed various tumor cells synergistically.

### Example 3: Effect of EGFR Inhibitor+TLR4/MD2 Pathway Inhibitor on the Growth of Cutaneous Melanomas in a Mouse Model

### 3.1 Experimental Materials

Experimental animals: Male BALB/c-nu/nu nude mice (about 8 weeks old), provided by the Animal Experiment Center of the Chinese University of Hong Kong, and housed in the Experimental Animal Center of Hong Kong Baptist University in a sterile environment with constant temperature and pressure.

A375 cell strain (human cutaneous melanoma cells carrying wild-type EGFRs): purchased from American Type Culture Collection (ATCC), and stored in liquid nitrogen.

Gefitinib intragastric solution: weigh 20 mg Gefitinib, add 20 mL solvent (2% Tween 80 PBS), and sonicate the mixture to obtain a Gefitinib suspension. Then the Gefitinib suspension was mixed to a homogeneous state, and sub-packaged and stored separately.

TAK-242 injection solution: weigh 10 mg TAK-242 and dissolve it into 0.5 mL DMSO. Then slowly diluted the solution with sterile PBS solution to a total volume of 5 mL to obtain 2 mg/mL TAK-242 injection solution. Then the solution was filtered, sub-packaged and stored separately.

### 3.2 Experimental Method and Result

A375 cells were cultured as described in Example 1.

The cultured A375 cells were collected, resuspended in phosphate buffered saline (PBS), and inoculated subcutaneously in the backs of BALB/c-nu/nu nude mice, wherein 3*10⁶ cells were injected into each mouse, the day of injection was marked as Day 0. On the 4th day, the mice were randomly grouped into 4 groups (n=4) by tumor volume (˜100 mm3): a control group, a Gefitinib group (positive control), a TAK-242 group and a drug combination group. The drugs were administered to the mice after fasting for 3 hours. The administration volume was calculated based on body weight, using a baseline of 100 μL injected and 200 μL gavaged per 20 g of body weight. In the Gefitinib group, mice were gavaged with 10 mg/kg Gefitinib and intraperitoneally injected with 100 μL of control vehicle (PBS containing 10% DMSO) every three days. In the TAK-242 group, mice were intraperitoneally injected with 10 mg/kg TAK-242 and gavaged with 200 μL of control vehicle (PBS containing 2% Tween 80) every three days. In the drug combination group, mice were gavaged with 10 mg/kg Gefitinib and intraperitoneally injected with 10 mg/kg TAK-242 every three days. In the control group, mice were intraperitoneally injected with 100 μL of control vehicle and gavaged with 200 μL of control solvent every three days. The drug administration lasted for 15 days.

The length and width of the mice's tumors were measured every 2-3 days, and the mice's body weights were weighed daily to plot curves of body weight changes and tumor volume changes. On day 20, the mice were euthanized, the solid tumors were dissected, weighed, and photographed.

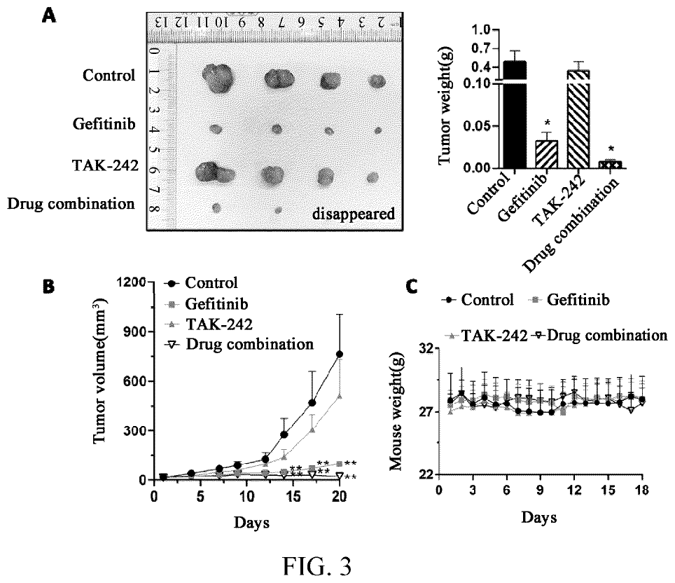

The experimental results are shown in FIG. 3. The results indicated that at the end of the experiment on day 20, the tumors in the Gefitinib group and the drug combination group were significantly smaller than those in the control group (p<0.05, FIG. 3A), and the tumor weight in the drug combination group was lower than that in the Gefitinib alone group or TAK-242 alone group (FIGS. 3A, 3B). It is particularly noteworthy that one tumor in the drug combination group had completely disappeared by day 20 (FIG. 3A). The drug treatments in each group had no significant effect on the body weight of the mice (FIG. 3C), indicating that the combination of EGFR inhibitor and TLR4/MD2 pathway inhibitor can safely and effectively inhibit the growth of cutaneous melanoma in mice, with a tumor inhibition rate significantly higher than that of EGFR inhibitor or TLR4/MD2 pathway inhibitor alone.

### Example 4. Effect of EGFR Inhibitor+TLR4/MD2 Pathway Inhibitor on the Growth of Non-Small Cell Lung Cancer and Gefitinib-Resistant Tumor in Mouse Model

### 4.1 Experimental Materials

Experimental animals: Male BALB/c-nu/nu nude mice (about 8 weeks old), purchased from Jiangsu GemPharmatech Co., Ltd., and raised in the Experimental Animal Center of Hong Kong Baptist University in a sterile environment with constant temperature and pressure.

PC9-GR cell strain (PC9 non-small cell lung cancer cells resistant to Gefitinib): purchased from the Cell Bank of China Academy of Sciences, and stored in liquid nitrogen.

Gefitinib oral gavage solution: weigh 15 mg Gefitinib, and add 20 mL solvent (2% Tween 80 PBS) to it. The mixture was ultrasonically treated to obtain a Gefitinib suspension. Then the Gefitinib suspension was mixed to a homogeneous state, and sub-packaged and stored separately.

TAK-242 injection solution: weigh 10 mg TAK-242 and dissolve into 0.5 mL DMSO. Then slowly dilute the solution with sterile PBS solution to 5 mL to obtain 2 mg/mL TAK-242 injection solution. Then the resultant solution was filtered, sub-packaged and stored separately.

### 4.2 Experimental Method and Result

The cell culture process was as described in Example 1. 1.8 M Gefitinib was added to the prepared DMEM medium to maintain drug resistance of the cells.

Cultured PC9-GR cells were collected, resuspended in phosphate-buffered saline (PBS), and then inoculated subcutaneously into the back of BALB/c-nu/nu nude mice, with 2×10⁵ cells injected per mouse, which was recorded as day 0. Four days after cell injection, the mice were randomly divided into four groups: vehicle control group (vehicle: 40% PEG400+5% Tween 20+45% saline+5% DMSO, administered via i.g. and i.p.); Gefitinib group (Gefitinib 10 mg/kg, positive control, administered via i.g.); TAK242 group (TAK242 10 mg/kg, administered via i.p.); and drug combination group (Gefitinib 10 mg/kg, administered via i.g. +TAK242 10 mg/kg, administered via i.p.); Subsequent administrations were performed once every three days, totaling six times. i.g. means intragastric administration; and i.p. means intraperitoneal injection.

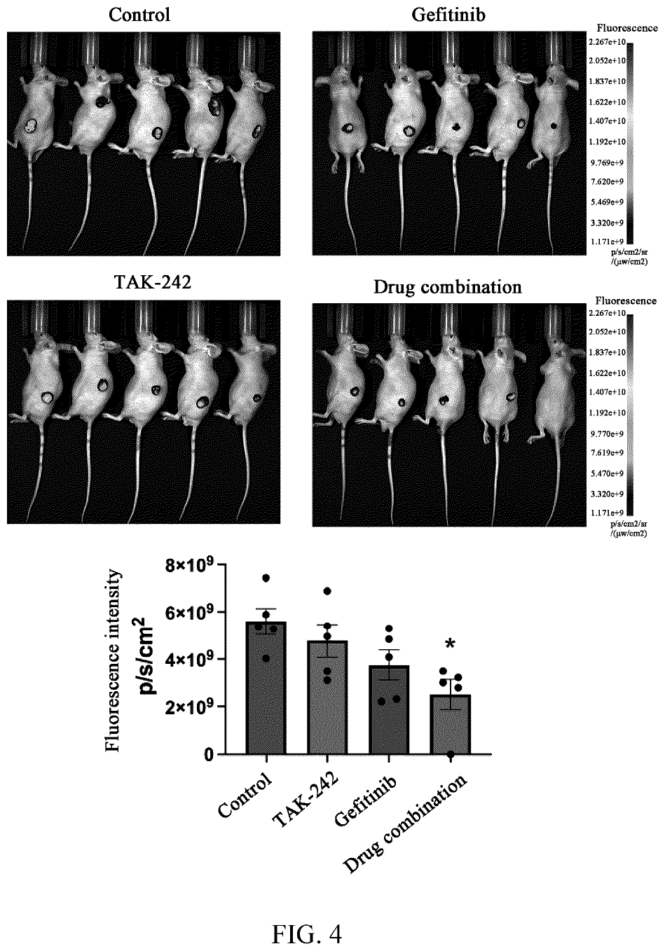

The experimental result is shown in FIG. 4. The result indicates that the tumor volume in the drug combination group was the smallest. The ratios of the average fluorescence intensity of the TAK-242 group, Gefitinib group, and combination treatment group to that of the control group were 0.85, 0.67, and 0.45, respectively. It is worth noting that the tumor in one mouse in the drug combination group completely regressed.

### Example 5. Drug Resistance in Tumor-Bearing Mice Treated with the Combination of EGFR Inhibitor and TLR4/MD2 Pathway Inhibitor

### 5.1 Experimental Materials

Experimental animals: Male BALB/c-nu/nu nude mice (about 8 weeks old), purchased from Jiangsu GemPharmatech Co., Ltd., and housed in the Experimental Animal Center of Hong Kong Baptist University in a sterile environment with constant temperature and pressure.

A375 cell strain (human cutaneous melanoma cells carrying wild-type EGFRs): purchased from American Type Culture Collection (ATCC), stored in liquid nitrogen.

Gefitinib oral gavage solution: weigh 15 mg Gefitinib, add 20 mL solvent (2% Tween 80 PBS) to it. The mixture was ultrasonically treated to obtain a Gefitinib suspension, and then the Gefitinib suspension was mixed to a homogeneous state, and sub-packaged and stored separately.

L48H37 injection solution: weigh 10 mg L48H37 and dissolve into 0.5 mL DMSO, then slowly dilute the solution with a sterile PBS solution to 5 mL to obtain 2 mg/mL L48H37 injection solution. The resultant solution was filtered, sub-packaged and stored separately.

### 5.2 Experimental Method and Result

Cultured A375 cells were collected, resuspended in phosphate-buffered saline (PBS), and inoculated subcutaneously into the backs of BALB/c-nu/nu nude mice at a dose of 2×10⁵ cells per mouse, which was recorded as day 0. On day 1, the mice were randomly divided into three groups by body weight: the gefitinib group (n=6), L48H37 group (n=7), and combination group (n=7). After a 3-hour fasting period, the mice were administered the respective treatments. Body weights were measured daily. The administration volume was calculated based on body weight using a baseline of 100 μL for injection and 200 μL for gavage per 20 g body weight. In the Gefitinib group, mice received 7.5 mg/kg Gefitinib via oral gavage and 100 μL of control solvent (PBS containing 10% DMSO) via intraperitoneal injection every three days; In the L48H37 group, mice received 10 mg/kg L48H37 via intraperitoneal injection and 200 μL of control vehicle (PBS containing 2% Tween 80) via oral gavage every three days; In the drug combination group, mice received 7.5 mg/kg gefitinib via oral gavage daily and 10 mg/kg L48H37 via intraperitoneal injection every day. The drug administration lasted for 37 days.

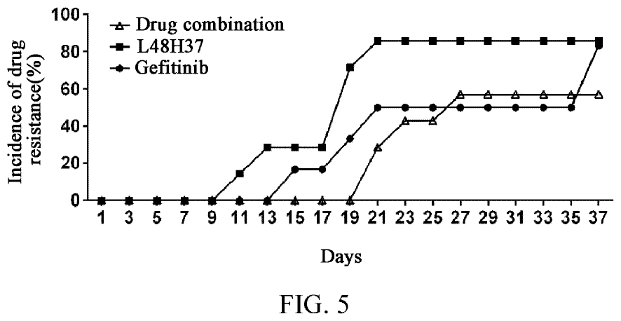

The length and width of the tumor in each mouse were measured once every 2-3 days, and the tumor volume was calculated. The tumor growth rate was calculated according to the tumor volume: growth rate=(tumor volume on Day 3)/(tumor volume on Day 1). If the tumor growth rate was lower than or equal to 1.3, it was suggested that the mice were sensitive to the drugs; otherwise, it was suggested that the mice had developed drug resistance. The experimental results are shown in Table 1 and FIG. 5.

TABLE 1
Drug Resistance Ratio of Tumor-Bearing Mice
to Gefitinib, L48H37 and Their Combination

| | Group | | |
|---|---|---|---|
| | | | Drug |
| Days | Gefitinib | L48H37 | Combination |
| 1 | 0/6 | 0/7 | 0/7 |
| 3 | 0/6 | 0/7 | 0/7 |
| 5 | 0/6 | 0/7 | 0/7 |
| 7 | 0/6 | 0/7 | 0/7 |
| 9 | 0/6 | 0/7 | 0/7 |
| 11 | 0/6 | 1/7 | 0/7 |
| 13 | 0/6 | 2/7 | 0/7 |
| 15 | 1/6 | 2/7 | 0/7 |
| 17 | 1/6 | 2/7 | 0/7 |
| 19 | 2/6 | 5/7 | 0/7 |
| 21 | 3/6 | 6/7 | 2/7 |
| 23 | 3/6 | 6/7 | 3/7 |
| 25 | 3/6 | 6/7 | 3/7 |
| 27 | 3/6 | 6/7 | 4/7 |
| 29 | 3/6 | 6/7 | 4/7 |
| 31 | 3/6 | 6/7 | 4/7 |
| 33 | 3/6 | 6/7 | 4/7 |
| 35 | 3/6 | 6/7 | 4/7 |
| 37 | 5/6 | 6/7 | 4/7 |
| Incidence of Drug Resistance (%) | 83.33 | 85.71 | 57.14 |

Note: The drug resistance ratio is calculated as the number of drug-resistant animals divided by the total number of animals in each group the incidence of drug resistance (%) is the drug resistance ratio multiplied by 100%.

The results showed that the onset time of drug resistance in tumor-bearing mice of the combination group (day 21) was later than that in the gefitinib group (day 15) and the L48H37 group (day 11). Moreover, the incidence of drug resistance at the end of the experiment in the combination group (57.14%) was lower than that in the gefitinib group (83.33%) and the L48H37 group (85.71%). These results indicate that compared with the single use of EGFR inhibitor or TLR4/MD2 pathway inhibitor, their combination can significantly delay and reduce the occurrence of drug resistance in tumor-bearing mice.

The above description of the embodiments is intended to facilitate those having ordinary skills in the art to understand and practice the present disclosure. It is obvious that those skilled in the art can easily make various modifications to those embodiments and apply the general principle described herein to other embodiments without expending any creative labor. Therefore, the present disclosure is not limited to the specific embodiments disclosed herein, and the improvements and modifications made by those skilled in the art according to the principle of the present disclosure without departing from the scope of the present disclosure should be deemed as falling in the scope of protection of the present disclosure.

What is claimed is:

1. A composition comprising a TLR4/MD2 pathway inhibitor and an EGFR inhibitor.

2. The composition according to claim 1, wherein the TLR4/MD2 pathway inhibitor is selected from chemical inhibitors, nucleic acid inhibitors and antibody inhibitors that inhibit the activation of a TLR4/MD2 pathway.

3. The composition according to claim 2, wherein the chemical inhibitors include small molecule inhibitors, such as TAK-242, LPS-RS, E5564, L48H37, MD2-IN-1, or L6H21;
wherein the nucleic acid inhibitors include nucleic acid molecules, such as siRNAs, miRNAs or antisense RNAs; and/or
wherein the antibody inhibitors include monoclonal antibodies, such as anti-TLR4 monoclonal antibodies or anti-MD2 monoclonal antibodies.

4. The composition according to claim 1, wherein the EGFR inhibitor is selected from chemical inhibitors, nucleic acid inhibitors and antibody inhibitors that inhibit EGFR.

5. The composition according to claim 4, wherein the chemical inhibitors include small molecule inhibitors, such as Gefitinib, Erlotinib, Afatinib, Osimertinib, Dacomitinib, Almonertinib, or Icotinib hydrochloride;
wherein the nucleic acid inhibitors include nucleic acid molecules, such as siRNAs, miRNAs or antisense RNAs; and/or
wherein the antibody inhibitors include monoclonal antibodies, such as Cetuximab, Panitumumab or Necitumumab.

6. The composition according to claim 1, wherein the EGFR inhibitor comprises Gefitinib, and the TLR4/MD2 pathway inhibitor comprises TAK-242 and/or L48H37.

7. The composition according to claim 1, wherein a mass ratio of the EGFR inhibitor to the TLR4/MD2 pathway inhibitor is about 1:(0.5-3).

8. The composition according to claim 1, comprising a first composition and a second composition, wherein the first composition comprises a TLR4/MD2 pathway inhibitor, and the second composition comprises an EGFR inhibitor, wherein the first composition and/or the second composition comprise(s) pharmaceutically acceptable excipients.

9. The composition according to claim 8, wherein the first composition comprises a small molecule TLR4/MD2 pathway inhibitor; and/or
the second composition comprises a small molecule EGFR inhibitor.

10. The composition according to claim 1, wherein the TLR4/MD2 pathway inhibitor is TAK-242 or L48H37, and the EGFR inhibitor is Gefitinib.

11. The composition according to claim 10, wherein a mass ratio of the Gefitinib to the TAK-242 is about 1:1 to 1:1.5; and/or
a mass ratio of the Gefitinib to the L48H37 is about 1:1 to 1:1.5.

12. A pharmaceutical composition comprising the composition according to claim 1 and optionally pharmaceutically acceptable excipients.

13. The pharmaceutical composition according to claim 12, wherein the pharmaceutical composition further comprises an additional anticancer therapeutic agent, which is selected from immunotherapeutic agents, chemotherapeutic agents, antiangiogenic agents, multidrug resistance-related protein inhibitors, radiotherapeutic agents, and any combination thereof.

14. A method of treating cancer, wherein the method comprises administering a therapeutically effective amount of the composition according to claim 1 to a subject in need thereof.

15. The method according to claim 14, wherein the cancer is selected from melanomas, eye cancers, oral cancers, lung cancers, liver cancers, bone cancers, brain cancers, gastrointestinal cancers, pancreatic cancers, neurological cancers, urogenital cancers, gynecological cancers, thyroid cancers, adrenal cancers, leukemias, lymphomas, and breast cancers.

16. The method according to claim 15, wherein the cancer is of a cancer type that is resistant to EGFR inhibitors.

17. The method according to claim 15, wherein the melanomas are selected from cutaneous melanomas and non-cutaneous melanomas;
the lung cancers are selected from non-small cell lung cancer and small cell lung cancer;
the gastrointestinal cancers are selected from esophageal cancer, gastric cancer, duodenal cancer, small intestine cancer, colon cancer, rectal cancer, colorectal cancer, anal cancer, gallbladder cancer, and cholangiocarcinoma;
the neurological cancers are selected from glioma, meningioma and neurilemmoma;
the urogenital cancers are selected from renal cancer, bladder cancer, urethral cancer, and prostatic cancer; and/or
the gynecological cancers are selected from cervical cancer, endometrial cancer, ovarian cancer, fallopian tube cancer and vaginal cancer.

18. The method according to claim 14, wherein the composition is administered by one or more of oral administration, percutaneous administration, intramuscular administration, subcutaneous administration, intraperitoneal administration, and intravenous administration.

19. The method according to claim 14, wherein the composition is in a dosage form of tablet, capsule, powder, injection, solution, suspension, emulsion, or any combination thereof.

20. The method according to claim 14, wherein the subject is a human or a non-human mammal, including primate such as monkeys, chimpanzees or gorillas; rodent such as mice or rats; bovid; equid; caprid; or canid.
